(19) United States
(12) Patent Application Publication
(10) Pub. No.: US 2008/0227984 A1
(43) Pub. Date: Sep. 18, 2008

(54) Selective Oxidation of Organic Compounds

(75) Inventors: Michael John Greenhill-Hooper, Miradoux (FR); Robert Raja, Southampton (GB); John Meurig-Thomas, Cambridge (GB)

Correspondence Address: SHERIDAN ROSS PC, 1560 BROADWAY, SUITE 1200, DENVER, CO, 80202, US

(73) Assignee: U.S. Borax, Inc., Englewood, CO (US)

(21) Appl. No.: 11/577,696

(22) PCT Filed: Oct. 21, 2005
(86) PCT No.: PCT/GB05/04062

§ 371 Date: Nov. 9, 2007

(30) Foreign Application Priority Data
Oct. 22, 2004 (GB) 0423586.7

(51) Int. Cl. C07D 213/807; C07C 51/31; C07D 301/03; C07C 37/00; C07D 307/33; C07C 17/013

(52) U.S. Cl. 546/327; 562/512.4; 549/523; 568/771; 549/295; 570/203

(57) ABSTRACT

This invention relates to the selective oxidation of organic compounds. According to the invention organic compounds are selectively oxidized using a peracid or a source of peracid, a transition metal based heterogeneous catalysts and a borate or boric acid in the presence of water. Using the process of the present invention, both excellent conversion and product selectivity maybe obtained.

This invention relates to the selective oxidation of organic compounds.

The selective oxidation of organic compounds is practised widely in the chemicals industry. The production of many large volume and high value fine chemicals employ oxidation reactions.

A list of industrially important chemical conversions falling within the category of oxidations includes: the epoxidation of olefins, the conversion of alkanes to alcohols, aldehydes, ketones and carboxylic acids, the Baeyer-Villiger oxidation of ketones to esters and lactones, the oxidation of alcohols to aldehydes, ketones and carboxylic acids, and the hydroxylation and oxyhalogenation of aromatics. Commercially important compounds like phenol, ethylene oxide (and ethylene glycol), propylene oxide (and propylene glycol), styrene oxide, caprolactone, adipic acid, catechol, hydroquinone, cresols, terpenoids, benzaldehyde, benzoic acid and chlorotoluenes all rely on oxidation reactions for their production.

Known chemical oxidation processes for the preparation of bulk chemicals on an industrial scale typically suffer from drawbacks, notably because they involve multi-step reactions, the use of expensive homogeneous catalysts that require costly separation and recycling steps, the use of atom-inefficient processes, they produce significant amounts of by-products or employ aggressive oxidants that in turn produce environmentally damaging waste products and emissions. For example, the currently practised route for producing phenol from benzene, involves a multi-step process whereby benzene is first converted to cumene, which is in turn oxidised to cumene hydroperoxide, and finally converted to the desired phenol, but with acetone as by-product. While direct routes have been proposed, such as oxidation of benzene using solid catalysts such as titanosilicates in combination with hydrogen peroxide and oxidation of benzene in the gas phase, these suffer from practical problems for commercial scale production.

Likewise, the commercial production of adipic acid from cyclohexane is a multi-step process using a soluble cobalt catalyst to produce cyclohexanol/cyclohexanone (“KA Oil”) in a first step, and subsequently further oxidising in the presence of concentrated nitric acid and a soluble vanadium catalyst to produce adipic acid but also 2 moles of the greenhouse gas nitric oxide for every mole of adipic acid.

Oxidation can also naturally be employed in the production of fine chemicals. Fine chemicals are generally prepared on a smaller scale than bulk chemicals such as discussed above. Nevertheless acceptable levels of selectivity are important, as are comparatively mild reaction conditions e.g. avoiding aggressive oxidation. In this connection particular mention may be made of the oxidation of heterocyclic aromatics such as alkyl pyridines. For example, 4-picoline, when oxidised at the methyl group, yields 4-picolinic or isonicotinic acid, which is an important derivative in the production of antibacterials, pharmaceuticals e.g. for the treatment of tuberculosis, psoriasis and arthritis (Isoniazid is isonicotinic acid hydrazide), as plant growth regulators, herbicides, pesticides and corrosion inhibitors. Similarly 3-picoline when oxidized at the methyl group yields nicotinic acid which is used in the preparation of vitamins (e.g. vitamin B3). While catalytic oxidations for such preparations have been proposed, the reactions in practice employ aggressive conditions such as provided by nitric acid, chromic acid and hydrobromic acid. One commercial route involves a two-step reaction via the corresponding 3-cyanopyridine.

There have recently been various studies on the use of oxidants with heterogeneous/solid catalysts, in particular heterogeneous transition metal catalysts, to provide more efficient selective oxidation processes for the preparation of organic compounds in solvent based systems. Such catalysts can be readily separated from the reaction media and recycled. In particular, it is believed that with the appropriate catalyst, there may be provided control of the site of oxidation of the starting material thus leading to good selectivity for the desired product and reducing the production of undesired by-products.

For example U.S. Pat. No. 5,767,320 describes the oxidation of cyclohexane to a mixture of cyclohexanone and cyclohexanol in the presence of an organic solvent with molecular oxygen in the presence of a solid catalyst containing a phthalocyanine or porphyrin complex of a transition metal where some or all of the hydrogen atoms of the transition metal complex have been substituted by one or more electron withdrawing groups. There is preferably present an alkyl hydroperoxide or dialkyl peroxide as promoter. The porphyrin complexes, encapsulated in zeolite are further described in Barley et al, New J Chem 1992, vol 16, page 71.

The oxidant used in such heterogeneous systems is generally molecular oxygen, preferably in the presence of a peroxide as promoter. It is also known to use hydrogen peroxide as an oxidant in oxidising reactions. Much work in the last two decades has been directed towards the use of hydrogen peroxide and molecular oxygen as oxidants in selective reactions. These are generally regarded as more mass efficient than many other oxygen sources.

It is also been proposed to use peracids, particularly peracetic acid, as oxidants in the oxidation of chemical compounds in solvent-based homogeneously catalysed. systems.

Despite the advances in heterogeneous catalysis, however, the yields of target products remain relatively low in some commercially important oxidation reactions using these catalysts and the aforementioned oxidants.

U.S. Pat. No. 5,462,692 describes solid acetyl peroxyborate compounds, which are active oxygen containing compounds, and their preparation from acetic acid and boron-oxygen compounds. The solid acetyl peroxyborates have a peracetic acid content which can be liberated together with hydrogen peroxide in water. The solid acetyl peroxyborates are proposed for use in washing, bleaching and cleaning agents and disinfectant applications and as oxidising agents, although there has been little practical use of these compounds to date.

The present invention provides a process for the selective oxidation of an organic compound which process comprises oxidising the organic compound using a peracid or a source of peracid, a transition metal based heterogeneous catalyst and a borate or boric acid, in the presence of water.

It has surprisingly been found that, under the conditions of the present invention, both excellent conversion and product selectivity may be obtained. Moreover the process achieves this using water as solvent and employing relatively mild conditions.

The process of the present invention may be applied to industrially important chemical conversions falling within the category of oxidations including: the epoxidation of olefins, the conversion of alkanes to alcohols, aldehydes, ketones and carboxylic acids, the Baeyer-Villiger oxidation of ketones to esters and lactones, the oxidation of alcohols to aldehydes, ketones and carboxylic acids, and the hydroxylation and oxyhalogenation of aromatics. Thus commercially important compounds like phenol, ethylene oxide (and ethylene glycol), propylene oxide (and propylene glycol), styrene oxide, caprolactone, adipic acid, catechol, hydroquinone, cresols, terpenoids, benzaldehyde, benzoic acid and chlorotoluenes can be prepared by the process of the present invention.

From the perspective of the fine chemical industry, the present invention is likely to be considered much safer because of its use of water as a solvent and the fact that air or oxygen is avoided.

Organic compounds that can be selectively oxidised using the process of this invention include substituted and unsubstituted aromatic hydrocarbons, olefins, alkanes, ketones and alcohols. The aromatics include benzene, phenol and toluene. Benzene may be oxidised to produce phenol, phenol may be oxidised to produce catechol and hydroquinone and toluene may be oxidised to benzaldehyde, o-cresol and p-cresol. In the presence of halide salts, for example sodium chloride, toluene may be converted using the process according to the invention to a mixture of o-, and p-chlorotoluene. The olefins include styrene, propylene, α-pinene, and (+)-limonene. These may all be converted to the corresponding epoxides, with minimal diol formation. The alkanes include cyclohexane, which may be converted to adipic acid, rather than the intermediate mixture of cyclohexanol and cyclohexanone produced by many existing processes. Cyclohexanone is an example of a ketone which may be oxidised in this process to the corresponding lactone,

For example the process according to the invention may be used for the oxidation of cyclohexane to adipic acid, the epoxidation of styrene to styrene oxide, oxidation of α-pinene to α-pinene-oxide, propylene to propylene oxide, oxidation of phenol to catechol and hydroquinone, oxidation of cyclohexanone to γ-caprolactone, oxidation of benzene to phenol and oxyhalogenation/chlorination of toluene to o- and p-chlorotoluene.

The selective oxidation of the present invention may also be used in the preparation of fine chemicals. In this connection particular mention may be made of alkyl e.g. methyl pyridines and heterocyclic aromatics e.g. nitrogen-containing aromatics, such as picolines, and alkyl, e.g. methyl, derivatives of pyrimidine, pyridazine, pyrazine, quinoline and quinoxaline. Using the process of the present invention such compounds may be converted into the corresponding carboxylic acids. Thus for example 4-picoline may be selectively converted into 4-picolinic acid (isonicotinic acid), 3-picoline to 3-picolinic acid (nicotinic acid) and alkyl quinolines to quinoline carboxylic acids and alkyl pyridazines to pyridazine carboxylic acids.

Isonicotinic acid may be used in the preparation of antibactierals, pharmaceuticals, plant growth regulators, herbicides, pesticides and corrosion inhibitors. Nicotinic acid may be used in the preparation of vitamins (e.g. vitamin B3). Quinoline carboxylic acids have possible uses in biocides, pesticides, antibacterials, cancer drugs, seed disinfectants, herbicides (e.g. Quinclorac, Imazaquin), plant growth regulators, antibiotics, antifungal agents for plants, trypsin inhibitors, charge control agents for photocopier toners, metal ion chelators for use in plating baths. Pyridazine carboxylic acids have uses in plant growth regulators e.g. Clofencet MON21200 ex Monsanto.

The peracid, which functions as oxidant in the process of the present invention, can be selected from any aliphatic or aromatic, generally carboxylic, peracid, including, but not limited to performic, peracetic, perpropionic, percaproic, pernonanoic, trifluoroperacetic and perbenzoic acid, and mixtures of peracids. Also included are solid forms of peracids, notably the acetylperoxyborate compounds described in U.S. Pat. No. 5,462,692, capable of releasing peracetic acid when dissolved in water.

Furthermore, there may be provided a source of peracid; that is the peracid can be formed in situ, for example by the reaction of the corresponding carboxylic acid, acid anhydride or acid chloride with hydrogen peroxide, or sources of hydrogen peroxide. The peracid can also be formed from the autoxidation of the appropriate aldehyde such as benzaldehyde. It is also possible to react hydrogen peroxide, or a source of hydrogen peroxide, with acyl-donating compounds, for example tetraacetylethylene diamine (TAED), tetraacetylglycoluril (TAGU) and sodium p-isononanoyloxy-benzenesulphonate (iso-NOBS). Sources of hydrogen peroxide include, but are not limited to sodium perborate monohydrate, sodium perborate tetrahydrate and sodium percarbonate.

The peracid component and the borate component may be combined. Thus for example when solid acetylperoxyborate compounds as described in U.S. Pat. No. 5,462,692 are dissolved in water they release, in addition to the peracid, peracetic acid, borate. Also when there is provided a source of peracid and one of the components used is a perborate for example as indicated above, that component can provide all or some of the borate.

There may further be used, as peracid or source thereof and source of borate, a mixture of compounds capable of reacting to form acetylperoxyborate. While acetylperoxyborate may itself not be formed in situ, the components in the appropriate ratio to form acetylperoxyborate may be used.

There may also be used, as the borate component present in the oxidation according to the invention, boric acid, metal borates and ammonium borates. The metal borates and ammonium borates may be selected from sodium perborate monohydrate, sodium perborate tetrahydrate, and borates with the general formula M₂O.xB₂O₃.yH₂O (with x ranging from 1 to 8, and y from 0 to 10), including disodium tetraborate penta- and decahydrate and forms of disodium tetraborate with lower levels of hydration, referred to as ‘puffed’ or ‘expanded’ borax, and sodium metaborate di- and tetrahydrate. M is preferably sodium but it can also represent ammonium and other alkali metals. Mixtures of borates can also be employed.

The transition metal based heterogeneous catalysts used in the process of the present invention include:

1. Transition metal substituted aluminophosphates (MeAlPOs), where the substituting metal Me can be for example Fe, Ru, Mn or Co and the AlPO framework structure includes, but is not limited to, AlPO 5, 18, 11 and 36. The aluminophosphates of the present invention used according to the invention may contain metal substitution levels in the range 0.02-0.10, and their preparation is described in for example S. T. Wilson, et al, J. Am. Chem.

Soc. 104 (1982) 1146; A. Simmen, et al, Zeolites 11(1991) 654; J. Chen, et al, J. Phys. Chem. 98 (1994) 10216 (AEI structure); R. Szostak, et al, in Synthesis of Microporous Materials, M. Occelli, H. Robson, (eds.), Van Nostrand Reinhold, New York (1992), pp 240 (AEL structure); J. M. Bennett, et al, ACS Symp. Series 218, Am. Chem. Soc., Washington, D.C., 1983, p. 109; S. T. Wilson, et al, U.S. Pat. No. 4,310,440 (1982); S. Oju, et al, Zeolites 9 (1989) 440 (AFI structure); P. A. Wright et al, Angew. Chem. Int. Ed. Engl., 31, 1472 (1992) (ATS structure).

Preferred transition metal substituted aluminophosphates used according to the invention have a pore size in the range 3.5 to 12 Angstroms and have some of the aluminium atoms replaced by transition metal atoms such as Fe, Ru, Mn, and Co, the AlPO framework structure being preferably of the form AlPO 5, 18, 11, or 36 and the transition metal substitution level being in the range of 2 to 10 atom percent.

2. Porphyrin and phthalocyanine transition metal complexes encapsulated within the pores of zeolites, typically super-cage zeolites, Na-X. For example the catalyst may be a solid catalyst containing a porphyrin or phthalocyanine complex of a transition metal wherein some or all of the hydrogen atoms of the complex have been replaced by electron withdrawing groups, the complex being encapsulated in a zeolite matrix as described in U.S. Pat. No. 5,767,320. Phthalocyanine-containing catalysts may for example be prepared by a process in which neat CuCl₁₆Pc, CoCl₁₆Pc and FeCl₁₆Pc are synthesized according to the procedure first reported by Birchall et al (J. Chem. Soc. C., 2667 (1970)) and modified by Raja and Ratnasamy (Appl. Catal. A., 158, L7 (1997)). The neat complexes may be encapsulated in the supercages of Faujasites (Zeolites Na-Y or Na-X) by the “zeolite synthesis method” reported by Balkus et al (Inorg. Chem., 33, 67 (1994)) and modified by Raja and Ratnasamy (Appl. Catal., 143, 145 (1996); Stud. Surf. Sci. Catal., 101, 181 (1996); J. Catal., 170, 244, (1997)). The “zeolite synthesis method” has a number of advantages (minimal amounts of free-metal or free-ligand, enhanced complex stability and minimal adsorption of complex on surface) over conventional “flexible-ligand” methods of encapsulation.

As indicated above there may be used transition metal complexes encaged in the super cage of zeolites Na-X. These include the phthalocyanine and porphyrin complexes described in U.S. Pat. No. 5,767,320. The transition metal in the complex may be selected from iron, cobalt, copper, chromium, manganese and mixtures thereof.

Preferred such catalysts are solid catalysts containing a phthalocyanine or porphyrin complex of a transition metal wherein some or all of the hydrogen atoms of the transition metal complex have been substituted by one or more electron withdrawing groups; the complex being encapsulated in a zeolite matrix.

3. Porous titanium-containing crystalline silicas comprising silicon and titanium oxides and known as titanium silicalites (e.g. TS-1). The products are synthesised by methods as described for example in U.S. Pat. No. 4,410,501.

The oxidation process of the present invention may be carried out with excellent selectivity. While it is not wished to be bound by theory, it is considered that the excellent selectivity is attributable in part to the 3-dimensional network form of the preferred catalysts used according to the invention. In particular it is believed the pore size of network controls the access and orientation of molecules to be oxidised and thus the selectivity of the reaction.

The catalysts used according to the invention generally have matrixes with pore sizes in the range 3.5 to 12 Angstroms.

The molar ratio of peracid, or peracid liberating component, or components employed, to the compound to be oxidised, is typically in the range 0.05:1 to 5:1, generally 0.05:1 to 3:1, e.g. 0.05:1 to 1:1, preferably 0.1:1 to 3:1, more preferably 0.1:1 to 0.4:1.

The amount of catalyst employed is typically in the range 1 to 20% by weight, preferentially in the range 2 to 15% by weight and further preferentially in the range 2 to 10% by weight, based on the weight of the compound being oxidised.

The weight ratio of borate, or borates to peracid employed is typically in the range 0.1:1 to 4:1.

The process according to the invention is carried out in the presence of water. This contrasts and is preferred to prior art processes carried out in organic solvents. The water present according to the invention may assist to liberate peracetic acid. Of course, there may also be present according to the invention an organic phase comprising e.g. substrate, products and optionally an inert water-immiscible organic solvent for solubilising the same, and the solid catalyst phase.

The temperature employed in the process varies according to the compound being oxidised, but is generally in the range from 25 to 120° C.

The reaction times employed also vary with the compound to be oxidised, but are generally within the range 0.2 to 20, e.g. 0.5 to 16, suitably 0.5 to 10, hours.

The reactions are generally carried out in a nitrogen purged atmosphere, and can be carried out under atmospheric pressure, providing that the temperature of the reaction does not exceed 100° C.

The invention is further illustrated with reference to the following Examples. The catalytic reactions were carried out in a stainless-steel catalytic reactor (100 ml, Parr) lined with Poly Ether Ether Ketone (PEEK). The substrates, a suitable internal standard (mesitylene) and catalyst were then introduced into the reactor and the reactor was sealed. The reactor and the inlet and outlet ports were purged with dry nitrogen prior to reaction. The contents of the reactor were stirred at 800 rpm and the reactor was heated to the desired temperature under autogeneous pressure (N₂).

The sources of peracid and borate (if separate from the source of the peracid) were dissolved in double distilled water and the resulting solution was fed slowly, over the course of the reaction, employing a syringe pump (Harvard “33”) to the stirred contents of the reactor.

Conversion (Conv) and selectivity (Sel) for each product were determined as defined by the following equations:

Conv %=[(moles of initial reactant−moles of residual reactant)/(moles of initial reactant)]×100

In most of the Examples, a ratio of compound being oxidised (substrate) to oxidant of 3:1 is employed. Thus the theoretical maximum conversion is 33.3%.

Sel %=[(moles of individual product)/(moles of total products)]×100.

### EXAMPLE 1

### Oxidation of Cyclohexane to Adipic Acid

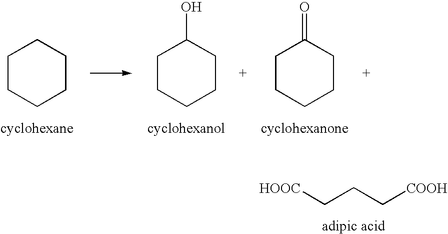

Two runs (each with different reaction times) were carried out as follows.

Solid acetylperoxyborate (3.49 g) prepared according to U.S. Pat. No. 5,462,692 and capable of liberating peracetic acid (0.701 g) and hydrogen peroxide (0.045 g) when dissolved in water, was mixed with double-distilled water (20 ml). The resulting solution was fed slowly by a syringe pump to a stirred reactor containing cyclohexane (2.5 g) and FeAlPO-31 catalyst (0.25 g), while the temperature was maintained at 110° C. This corresponds to a cyclohexane to peracetic acid molar ratio of 3:1.

The reaction products were analysed by gas chromatography (GC, Varian Model 3400 CX) employing a HP Innovax Column (30 m×0.53 mm×0.1 μm) and flame ionisation detector using a variable ramp temperature program from 65° C. to 220° C.

The identity of each product was first confirmed using authenticated standards and their individual response factors were determined using a suitable internal standard (calibration method).

The identity of the products was also confirmed by liquid crystal mass spectrometry using an LCMS-QP8000 (Shimadzu).

The reaction pH was 5.2.

One run was conducted for 16 hours. In this case the results were as follows:

Conversion of cyclohexane to oxidised products was calculated to be 29.5%.

### Product Selectivity was as Follows:

| Product | Selectivity (%) |
|---|---|
| Adipic acid | 81.2 |
| Cyclohexanone | 11.3 |
| Other acids* | 7.5 |

*Other acids (here and below) = succinic, glutaric and valeric acids.

One run was conducted for 8 hours. In this case the results were as follows:

Conversion of cyclohexane to oxidised products was calculated to be 24.7%.

### Product Selectivity was as Follows:

| Product | Selectivity (%) |
|---|---|
| Adipic acid | 67.0 |
| Cyclohexanone | 27.2 |
| Other acids | 5.8 |

### EXAMPLE 2

Two runs (each with a reaction time of 16 hours) were carried out a follows.

A liquid comprising borax pentahydrate (1.9 g) (Neobor ex Borax Europe Limited), sodium perborate monohydrate (0.4 g), 25% peracetic acid solution in acetic acid (4.2 g) and double-distilled water (20.5 g) was fed slowly by a syringe pump to a stirred reactor containing cyclohexane (2.5 g) and FeAlPO-31 catalyst (0.25 g), while the temperature was maintained at 110° C.

The Results were as Follows:

Conversion of cyclohexane to oxidised products was calculated to be 25.0% (Run 1) and 26.5% (Run 2).

### Product Selectivity was as Follows:

| Product | Run 1 (%) | Run 2 (%) |
|---|---|---|
| Adipic acid | 61.4 | 63.3 |
| Cyclohexanone | 21.5 | 19.3 |
| Cyclohexanol | 3.4 | 4.5 |
| Other acids | 12.0 | 11.0 |
| Carbon dioxide | 1.5 | 1.7 |

### EXAMPLE 3 (COMPARATIVE)

Two runs (each with a reaction time of 16 hours) were carried out a follows.

A solution containing 25% peracetic acid solution in acetic acid (4.2 g) and double-distilled water (20.5 g), was fed slowly by a syringe pump to a stirred reactor containing cyclohexane (2.5 g) and FeAlPO-31 catalyst (0.25 g), while the temperature was maintained at 110° C.

The reaction pH was 1.65.

The Results were as Follows:

Conversion of cyclohexane to oxidised products was calculated to be 32.5% (Run 1) and 32.3% (Run 2)

### Product Selectivity was as Follows:

| Product | Run 1 (%) | Run 2 (%) |
|---|---|---|
| Adipic acid | 30.5 | 33.1 |
| Cyclohexanone | — | — |
| Cyclohexanol | — | — |
| Other acids | 59.0 | 56.0 |
| Carbon dioxide | 10.3 | 10.5 |

### EXAMPLE 4 (COMPARATIVE)

The procedure of Example 1 was repeated but air was used as oxidant and the reaction was conducted for 24 hours. Full experimental details are given in M Dugal, G Sankar, R Raja & J M Thomas, Angew Chem Ed Engl, 39, 2310-2313 (2000).

Conversion of cyclohexane to oxidised products was only 6.6%.

### Product Selectivity was as Follows:

| Product | Selectivity (%) |
|---|---|
| Adipic acid | 65 |
| Cyclohexanone | 15.3 |

### EXAMPLE 5

The procedure of Example 1 was repeated but using, as oxidant-containing solution, (a) peracetic acid solution containing borax pentahydrate or (b) peracetic acid solution containing sodium acetate.

The oxidant solution (a) was obtained from 25% peracetic acid solution in acetic acid (4.2 g), Neobor (1 g), NaOH (1 g) and double-distilled water (20.5 g)

The oxidant solution (b) was obtained from 25% peracetic acid solution in acetic acid (4.2 g), sodium acetate trihydrate (0.934 g), NaOH (1 g) and double-distilled water (20.5 g).

Each run was conducted for 16 hours and the pH was, in each case, 5.1.

### Product Selectivity was as Follows:

| Product | Oxidant a (%) | Oxidant b (%) |
|---|---|---|
| Adipic acid | 72.5 | 51.2 |
| Cyclohexanone | 17.7 | 16.8 |
| Cyclohexanol | 3.3 | 3.5 |
| Other acids | 6.5 | 24.3 |
| Conversion | 27.5 | 29.9 |

### CONCLUSION

While Examples 1 and 2 according to the invention lead to acceptably high rates of both conversion and selectivity for the desired adipic acid, this was not the case with comparative Examples 3 and 4.

Example 5 demonstrates the contribution of the borate component to selectivity.

### EXAMPLE 6

### Epoxidation of Styrene to Styrene Oxide

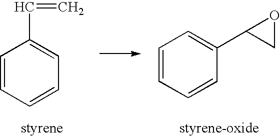

This example was carried analagously to the procedure of Example 1.

Solid acetylperoxyborate (3.49 g), capable of liberating peracetic acid (0.701 g) and hydrogen peroxide (0.045 g) when dissolved in water, was mixed with double-distilled water (20 ml). The resulting solution was fed slowly by a syringe pump to a stirred reactor containing styrene (2.8 g) and MnAlPO-5 catalyst (0.25 g), while the temperature was maintained at 65° C., and the reaction time was 1 hour.

The reaction pH was 5.2.

The reaction products were analysed by gas chromatography (GC, Varian Model 3400 CX) employing a HP-1 capillary column (25 m×0.32 mm) and flame ionisation detector.

The conversion of styrene was 31.7% and the selectivity for styrene oxide was 100%.

### EXAMPLE 7

This example was carried out analagously to the procedure of Example 2.

Two runs (each with a reaction time of 1 hour) were carried out a follows.

A liquid comprising borax pentahydrate (1.9 g) (Neobor ex Borax Europe Limited), sodium perborate monohydrate (0.4 g), 25% peracetic acid solution in acetic acid (4.2 g) and double-distilled water (20.5 g) was fed slowly by a syringe pump to a stirred reactor containing styrene (2.8 g) and MnAlPO-5 catalyst (0.25 g), while the temperature was maintained at 65° C.

The Results were as Follows:

Conversion of styrene was calculated to be 26.5% (Run 1) and 27.1% (Run 2).

### Product Selectivity was as Follows:

| Product | Run 1 (%) | Run 2 (%) |
|---|---|---|
| Styrene oxide | 88.7 | 89.0 |
| Diol | 11.3 | 11.0 |

### EXAMPLE 8 (COMPARATIVE)

This example was carried out analagously to the procedure of Example 3.

Two runs (each with a reaction time of 1 hour) were carried out a follows.

A solution containing 25% peracetic acid solution in acetic acid (4.2 g) and double-distilled water (20.5 g), was fed slowly by a syringe pump to a stirred reactor containing styrene (2.8 g) and MlnAPO-5 catalyst (0.25 g), while the temperature was maintained at 65° C.

The reaction pH was 1.65.

The Results were as Follows:

Conversion of styrene was calculated to be 32.5% (Run 1) and 32.3% (Run 2).

### Product Selectivity was as Follows:

| Product | Run 1 (%) | Run 2 (%) |
|---|---|---|
| Styrene oxide | 15.5 | 17.0 |
| Diol | 35.2 | 32.5 |
| Benzaldehyde | 39.7 | 41.2 |
| Polymers | 9.5 | 9.3 |

### EXAMPLE 9

The procedure of Example 6 was repeated but using, as oxidant containing solutions (a) peracetic acid solution containing borax pentahydrate as described in Example 5 or (b) peracetic acid solution containing sodium acetate as described in Example 5.

Each run was conducted for 1 hour and the pH was, in each case, 5.1.

### Product Selectivity was as Follows:

| Product | Oxidant a (%) | Oxidant b (%) |
|---|---|---|
| Styrene oxide | 87.3 | 63.3 |
| Diol | 12.5 | 27.5 |
| Benzaldehyde | — | 9.2 |
| Conversion | 24.3 | 26.0 |

### EXAMPLE 10

This example relates to the epoxidation of styrene to styrene oxide using air and benzaldehyde where the benzaldehyde is used as a sacrificial oxidant to produce perbenzoic acid in situ.

A liquid comprising borax pentahydrate (1.9 g) (Neobor ex Borax Europe Limited), sodium perborate monohydrate (0.4 g), benzaldehyde and double-distilled water (20 g) was fed slowly by a syringe pump to a stirred reactor containing styrene (35 g) and MnAlPO-5 catalyst (0.25 g) under air (dry; 30 bar). The styrene: benzaldehyde molar ratio was 1:3. The temperature was maintained at 50° C. and the reaction time was 4 hours.

The Results were as Follows:

Conversion of styrene was calculated to be 45.3%. (Theoretical maximum 100% as an excess of air and benzaldehyde was used).

### Product Selectivity was as Follows:

| Product | Selectivity (%) |
|---|---|
| Styrene oxide | 71.3 |
| Diol | 20.5 |
| Polymers | 8.1 |

### EXAMPLE 11 (COMPARATIVE)

Example 10 was repeated but omitting the borax pentahydrate, sodium perborate monohydrate and the distilled water.

The Results were as Follows:

Conversion of styrene was calculated to be 32.0%.

### Product Selectivity was as Follows:

| Product | Selectivity (%) |
|---|---|
| Styrene oxide | 49 |
| Diol | 50 |
| Polymers | 1 |

### CONCLUSION

While Examples 6, 7 and 10 according to the invention lead to acceptably high conversion and selectivity for the desired styrene oxide, this was not the case with comparative Examples 8 and 11.

Example 9 demonstrates the contribution of the borate component to selectivity.

### EXAMPLE 12

### Oxidation of α-Pinene

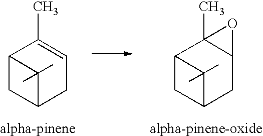

The procedure of Example 6 was repeated but using α-pinene (3.7 g), instead of the styrene, and using MnAlPO-5 (0.25 g) as catalyst. The reaction temperature employed was 65° C. and the reaction time was 1 hour.

Conversion was 25.9% and the selectivity for a-pinene-oxide was 100%.

### EXAMPLE 13

### Oxidation of Phenol

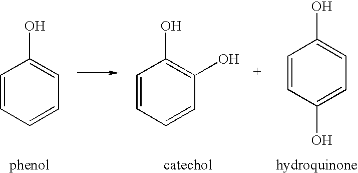

The procedure of Example 1 was repeated but using phenol (2.5 g), instead of the cyclohexane, and using iron hexa deca chloro phthalocyanine encapsulated in zeolite Na-X (0.25 g) as catalyst. The reaction temperature employed was 90° C. and the reaction time was 6 hours.

The reaction products were analysed by gas chromatography (GC, Varian Model 3400 CX) employing a HP-1 capillary column (25 m×0.32 mm) and flame ionisation detector.

Conversion was 31.5% and the selectivity was catechol 73.7% and hydroquinone 26.5%.

### EXAMPLE 14

The procedure of Example 13 was repeated but using FeAlPO-5 as catalyst.

Conversion was 27.6% and the selectivity was catechol 49.5% and hydroquinone 50.3%.

### EXAMPLE 15

### Oxidation of Cyclohexanone

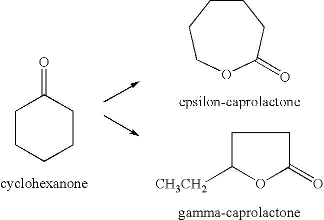

The procedure of Example 6 was repeated but using cyclohexanone (2.65 g), instead of the styrene, and using MnAlPO-5 (0.25 g) as catalyst. The reaction temperature employed was 50° C. and the reaction time was 2 hours.

Conversion was 31.5% and the selectivity for γ-caprolactone was 99.8%.

### EXAMPLE 16

### Oxidation of Benzene

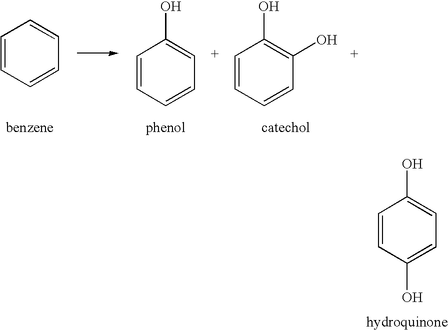

The procedure of Example 1 was repeated but using benzene (2.15 g), instead of the cyclohexane, and using copper hexa deca chloro phthalocyanine encapsulated in zeolite Na-X (0.25 g) as catalyst. The reaction temperature employed was 80° C. and the reaction time was 6 hours.

The reaction products were analysed by gas chromatography (GC, Varian Model 3400 CX) employing a HP-1 capillary column (25 m×0.32 mm) and flame ionisation detector.

Conversion was 11.5% and the selectivity for phenol was 95.2%.

### EXAMPLE 17

### Oxyhalogenation (Chlorination) of Toluene

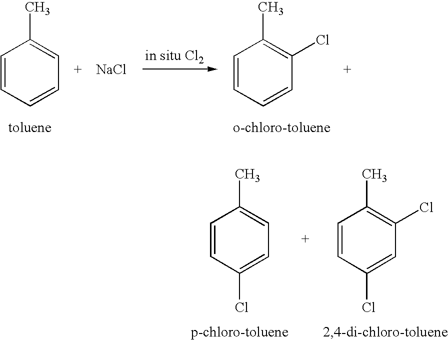

The procedure of Example 1 was repeated but using toluene (2.5 g), instead of the cyclohexane, and using iron hexa deca chloro phthalocyanine encapsulated in zeolite Na-X (0.25 g) as catalyst. NaCl (4.73 g dissolved in 10 ml water) was used as the source of halogen (toluene:NaCl molar ratio 1:3). The reaction temperature employed was 90° C. and the reaction time was 10 hours.

The reaction products were extracted with diethyl ether at the end of the reaction and were analysed by gas chromatography (GC, Varian Model 3400 CX) employing a HP-1 capillary column (25 m×0.32 mm) and flame ionisation detector.

Conversion was 32.5% and the selectivity was o-chlorotoluene 21.5% and p-chlorotoluene 78.4%.

### EXAMPLE 18

### Oxidation of 4-Picoline to 4-Picolinic Acid (Isonicotinic Acid)

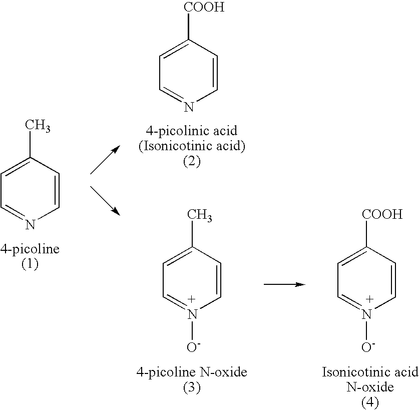

Solid acetylperoxyborate (3.49 g) as described in Example 1 was dissolved in double distilled water (20.5 g). The resulting solution was fed slowly by a syringe pump to a stirred reactor containing 4-picoline (2.8 g) and MnALPO-5 catalyst (0.30 g). This corresponds to a 4-picoline to peracetic acid molar ratio of 3:1.

Six runs (each of 4 hours) were carried out with different temperatures being maintained.

The Results Obtained were as Follows:

| | |
|---|---|
| Conversion | Product Selectivity (%) |

| Temp° C. | (%) | 2 | 3 | 4 | Others |
|---|---|---|---|---|---|
| 65 | 13.8 | 100 | — | — | — |
| 75 | 16.7 | 100 | — | — | — |
| 85 | 20.5 | 92.5 | 2.2 | — | 5.0 |
| 95 | 24.3 | 91.0 | 3.5 | — | 5.3 |
| 105 | 28.3 | 87.0 | 11.2 | — | 1.7 |
| 115 | 32.2 | 71.1 | 11.9 | 16.9 | — |

It can be seen that at 65° and 75° C. very good selectivity for the desired oxidation of the methyl group to a carboxylic acid was obtained.

At higher temperatures, while there is increased conversion, selectivity is decreased as in particular, increased oxidation of the ring nitrogen atom was observed.

### EXAMPLE 19

The procedure of Example 18 was followed with the exception that there were used as oxidant-containing liquid:

(i) borax pentahydrate (1.9 g) (Neobor), sodium perborate monohydrate (0.4 g), 25% peracetic acid solution in acetic acid (4.2 g) and double-distilled water (20.5 g);

(ii) peracetic acid solution as described in Example 3;

(iii) peracetic acid solution containing borax pentahydrate as described in Example 5;

(iv) peracetic acid solution containing sodium acetate as described in Example 5;

(v) hydrogen peroxide such that the 4-picoline to oxidant molar ratio was 3:1;

(vi) t-butyl hydroperoxide such that the 4-picoline to oxidant molar ratio was 3:1

Each run was carried out at 95° C. for 4 hours.

The Results Obtained were as Follows:

| | |
|---|---|
| Conversion | Product Selectivity (%) |

| Oxidant | (%) | 2* | 3* | 4* | Others |
|---|---|---|---|---|---|
| (i) | 22.8 | 88.3 | 5.5 | | 6.0 |
| (ii) | 24.8 | 44.1 | 20.5 | 25.9 | 9.3 |
| (iii) | 22.2 | 89.5 | 4.2 | 1.5 | 4.7 |
| (iv) | 27.1 | 33.7 | 45.3 | 15.5 | 5.4 |
| (v) | 32.1 | 63.0 | 27.0 | 8.5 | 1.3 |
| (vi) | 30.4 | 71.2 | 11.5 | 15.0 | 2.5 |

*See Example 18

The best selectivity of isonicotinic acid is obtained in the Examples where boron is present.

### EXAMPLE 20

The procedure of Example 18 was followed with the exception that the mole ratio of substrate to oxidant was varied. Each run was carried out at 95° C. for 4 hours.

| Substrate: | Conversion % | | | | |
|---|---|---|---|---|---|
| Oxidant | (Theoretical | Product Selectivity (%) | | | |
| (mole ratio) | max) | 2* | 3* | 4* | Others |
| 1:1 | 78.2 ₍₁₀₀₎ | 80.3 | 6.2 | 10.4 | 3.2 |
| 2:1 | 35.5 ₍₅₀₎ | 86.1 | 4.1 | 7.1 | 2.7 |
| 3:1 | 24.3 _{(33.3)} | 91.2 | 3.6 | 0 | 5.2 |
| 4:1 | 18.7 ₍₂₅₎ | 91.4 | 4.0 | 0 | 4.5 |
| 5:1 | 15.3 ₍₂₀₎ | 90.6 | 4.2 | 0 | 5.1 |

It can be seen that at low substrate to oxidant ratios (e.g. 1:1), good conversions and selectivities are obtained.

### EXAMPLE 21

The procedure of Example 18 was followed with the exception that there was used as catalyst TS-1 (titanium silicalite ex National Chemical Laboratory, Pune, India) (0.3 g).

Four runs (each of 4 hours) were carried out with different temperatures being maintained.

The Results Obtained were as Follows:

| | |
|---|---|
| Conversion | Product Selectivity (%) |

| Temp° C. | (%) | 2* | 3* | 4* | Others |
|---|---|---|---|---|---|
| 65 | 9.4 | 73.0 | 18.2 | 6.5 | 2.3 |
| 75 | 12.6 | 65.0 | 23.7 | 8.6 | 2.8 |
| 85 | 15.5 | 56.5 | 27.9 | 10.5 | 4.9 |
| 95 | 20.6 | 45.0 | 35.0 | 11.3 | 8.5 |

*See Example 18.

### EXAMPLE 22

The phases from the product of Example 6 were separated and the catalyst phase was calcined at 550° C. in air for 16 hours. The recovered catalyst was used in the procedure according to Example 6. Conversion of styrene was 30.0%. The catalyst was separated and re-calcined as described above. The recovered catalyst was again used in the procedure according to Example 6. Conversion of styrene was 29.9%. In both cases, selectivity was 100% for styrene oxide. This demonstrated that the catalyst was recyclable and maintained its activity.

1. A process for the selective oxidation of an organic compound which process comprises oxidising the organic compound using a peracid or a source of peracid, a transition metal based heterogeneous catalyst and a borate or boric acid, in the presence of water.

2. A process according to claim 1 wherein there is used, a peracid or source thereof and source of borate, acetylperoxyborate.

3. A process according to claim 1 wherein there is used, a peracid or source thereof and source of borate, a mixture of compounds capable of reacting together to form acetylperoxyborate.

4. A process according to claim 3 wherein the mixture of compounds comprises borax pentahydrate, sodium perborate monohydrate and peracetic acid.

5. A process according to claim 1 wherein there are used, a peracid or source thereof and source of borate, benzaldehyde, borax pentahydrate and sodium perborate in the presence of air.

6. A process according to claim 1 wherein the molar ratio of peracid or source thereof to the compound to be oxidised, is 0.05:1 to 3:1.

7. A process according to claim 1 wherein the weight ratio of borate or boric acid to peracid employed is in the range 0.1:1 to 4:1.

8. A process according to claim 1 wherein the transition metal based heterogeneous catalyst comprises a matrix having a pore size in the range 3.5 to 12 Angstroms.

9. A process according to claim 1 wherein the transition metal based heterogeneous catalyst used is a transition metal substituted aluminophosphate having a pore size in the range 3.5 to 12 Angstroms and having some of the aluminium atoms replaced by transition metal atoms, the transition metal substitution level being in the range 2 to 10 atom percent.

10. A process according to claim 1 wherein the transition metal based heterogeneous catalyst used comprises a phthalocyanine or porphyrin complex of a transition metal wherein some or all of the hydrogen atoms of the transition metal complex have been substituted by one or more electron withdrawing groups; the complex being encapsulated in a zeolite matrix.

11. A process according to claim 1 wherein the transition metal based heterogeneous catalyst used is titanium silicalite.

12. A process according to claim 1 wherein the amount of catalyst employed is in the range 1 to 20% by weight based on the weight of the compound being oxidised.

13. A process according to claim 1 for the selective oxidation of substituted and unsubstituted aromatic hydrocarbons, olefins, alkanes, ketones and alcohols.

14. A process according to claim 1 for the oxidation of cyclohexane to adipic acid, the epoxidation of styrene to styrene oxide, oxidation of α-pinene to α-pinene-oxide, oxidation of propylene to propylene oxide, oxidation of phenol to catechol and hydroquinone, oxidation of cyclohexanone to γ-caprolactone, oxidation of benzene to phenol and oxyhalogenation/chlorination of toluene to o- and p-chlorotoluene.

15. A process according to claim 1 for the selective oxidation of heterocyclic aromatics to yield corresponding carboxylic acids.

16. A process according to claim 1 for the selective oxidation of alkyl pyridines to yield corresponding carboxylic acids.

17. A process according to claim 1 for the selective oxidation of 4-picoline to 4-picolinic acid (isonicotinic acid).

18. (canceled)
